(19) United States
(12) Patent Application Publication
(10) Pub. No.: US 2010/0145185 A1
(43) Pub. Date: Jun. 10, 2010

(54) EXTENDING THE RESOLUTION OF MRI DATA BY COMBINING SUBSETS FROM PLURAL IMAGE ACQUISITIONS

(76) Inventors: Xiaole Hong, Acton, MA (US); Steven E. Harms, Fayetteville, AR (US); Aiping Jiang, Lexington, MA (US)

Correspondence Address: DUANE MORRIS LLP - Philadelphia;IP DEPARTMENT, 30 SOUTH 17TH STREET, PHILADELPHIA, PA, 19103-4196, US

(21) Appl. No.: 12/328,587

(22) Filed: Dec. 4, 2008

(51) Int. Cl. A61B 5/055

(52) U.S. Cl. 600/420; 600/410

(57) ABSTRACT

An MRI image from spiral trajectory scanning is arranged as complementary subsets of values in time-sampled k-space. These values are Fourier transformed to produce a spatial domain image. While holding the patient stationary, the contrast information is updated at the central portion of k-space, and the peripheral portion of k-space data can be filled during the whole image acquisition. The contrast information is combined with the peripheral portion of k-space (contributing to image resolution) to construct a full k-space data and to generate a spatial image. The technique is useful for providing short time interval sampling when analyzing the take-up and fade-away of a contrast agent over time.

### FIELD OF THE INVENTION

The invention relates to nuclear magnetic resonance imaging. Complementary subsets of k-space data from different MRI data acquisitions are combined, and the combinations are Fourier transformed to produce spatial images. A subset of k-space data from a central volume of k-space can be collected repeatedly in each successive MRI acquisition. The central k-space subset is combined with data from a peripheral volume in k-space that is collected less frequently, or even only once. Fourier transforming the combinations produces multiple images with a short acquisition time.

### BACKGROUND

Nuclear magnetic resonance imaging (NMR or MRI) relies on the relaxation properties of nuclei in imaged volumes of tissue, when subjected to a steady state magnetic biasing field, excited by radio frequency signals. The tissue is caused to produce responsive electromagnetic radiation at locations that are addressed by timed gradient magnetic fields. Volumetric image data is resolved from amplitude and phase information in responsive signals, that are digitized and mathematically processed.

One object of MRI is to collect data values that are resolved according to location in the imaged tissue so that tissue structures are distinguishable because the tissues contain different densities of detectable elements. Different relaxation times or other different chemical/physical properties may change the responsive MR signal strength and timing. Internal tissue structures can be visualized in processed images according to the local MR response. The images produced as outputs are mathematically processed representations of the electromagnetic response produced at spaced volume points in the tissue volume, ultimately represented by points or voxels in an image.

In the spatial domain, voxel values comprise a three dimensional matrix wherein at least one sensed or processed value for each voxel (such as the amplitude of spatially localized spin echo or gradient echo response) is represented by one or more of luminance, hue and saturation at a voxel location projected to a pixel position on a graphic display. The data can be represented as planar slices or as two dimensional projections of three dimensional volumes, wherein visual aspects such as grey scale, hue, color saturation/opacity and the like are made to vary with the concentrations of elements detected at corresponding voxel locations. Where successive images are acquired, the images can display certain changes over time. This is useful, for example, to show the progress of a contrast agent through vascular passages. In the case of a static image, the display can be arranged to change the manner of representation of static data, including for example, advancing a two dimensional display through successive slices, changing the magnification, rotating the image to view the volume from a different point of view.

When the magnetic resonance data is sensed and digitized, the information is in the time domain rather than the spatial domain. Imaging as described requires transforming time domain information to spatially resolved concentrations of elements having nuclei that resonate at different resonant frequencies. The information needs to be resolved down to the spatial resolution of a voxel in the image. Fourier transforms are applied to convert the time information from so-called k-space, through frequency and phase to spatially resolved local amplitudes.

“K-space” is a matrix of data values in three dimensions, collected by sampling and digitizing the MR response of the tissue being imaged. However, the values of the voxel data in image space are obtained by Fourier transforms applied to the k-space data set.

An excitation RF signal applied to tissues during imaging produces a response at local areas within the tissue depending on the composition of the tissue, the excitation frequency and timing. The response characteristic of one tissue type or two or more response characteristics may be collected and mapped or otherwise used together with one another to distinguish tissue types. Thus, resonant and off-resonant responses, spin echo and phase relaxation (T1 and T2) attributes, responses to Larmor frequencies for water versus fat, and similar responses are useful individually or in combinations for producing volumetric images of tissue composition elements and structures that assist in diagnoses and treatments.

For example, water concentration versus fat concentration distinguishes some tissue types. MRI can also distinguish circumstances affecting tissues, such as those that affect blood perfusion, e.g., edema or ischemia. Concentrations can differ by virtue of the presence or concentration of magnetic compounds such as iron nuclei in hemoglobin, which enables visualization of vasculature. Distinctions among tissues can be enhanced by perfusing tissue with a contrast agent such as a gadolinium compound. Some contrast agents bind to particular tissue structures such as tumors and lesions, and the rate of contrast agent's uptake and/or the rate of washout in different tissues over time, provide ways to distinguish among tissue types and/or circumstances that affect tissues.

The nuclei of atoms have magnetic moments that can become aligned when subjected to a biasing magnetic field. Application of a radio frequency pulse excitation signal at a resonant frequency for a particular element or isotope (the Larmor frequency) reorients the magnetic moments of the nuclei that correspond to that resonant frequency. The excitation tilts the nuclei relative to their biased alignment and in local areas that are selectable by varying magnetic field strength, the atoms precess (spin) in phase. Over a brief period of time after the excitation (e.g., tens of milliseconds), the phase coherence of the spins dissipates. Over a longer period (e.g. approximately a second), the nuclei return to their original biased magnetic moment alignments. The times associated with loss of phase coherence and alignment are distinct for different types of tissues and their environment. It takes time to establish gradient field conditions and to execute a sequence of excitation and sensing steps so as to obtain the MR response at particular tissue locations. Imaging a volume requires execution of many such sequences.

A salient use of nuclear magnetic resonance imaging is in diagnosis of lesions and tumors in breast tissue. For breast imaging, the MRI data advantageously is collected and processed to minimize the representation of fatty tissues that could obscure visualization of lesions. Perfusion with contrast agents improves the contrast further and also enables distinctions to be drawn among tissues based on different rates of diffusion of the contrast agent.

Various results are obtainable using different excitation pulse sequences to develop data wherein the encoded value for each voxel represents the local concentration and response of a particular element. The distinguishing parameters can be the amplitude and phase of spatially localized RF emission at a resonance frequency, the timing of the echo response, and other aspects that permit one element to be distinguished from another element and/or permit assessment of the relative signal intensities of elements at different locations, while varying gradient fields to move the lines along which data values are collected during each sequence.

As mentioned above, the magnetic resonance data when sensed and digitized provides amplitude samples at discrete sample times. A one-dimensional inverse Fourier transform of each echo produces a projection of the spin distribution along the read axis. A second inverse Fourier transform along the phase encoded axis provides a second dimension of spatial encoding.

The total sampling time is determined by the number of sampled points per read gradient and the number of phase-encode gradient steps. In a method wherein the field of view corresponds to a slice through the patient tissues in an X-Y plane, the relative position of patient might be advanced incrementally in a Z direction normal to the slice plane, whereupon a next parallel slice is imaged. The X-Y pixels in a slice, and the pixels of the successive slices, are interpreted together as a volume image wherein the voxel resolution is the resolution within the slices and the distance of incremental advance between slices.

Another method of MRI imaging uses spiral 3D acquisition wherein the excitation response encompasses multiple planes. The in-plane (X-Y) data is acquired by proceeding in a spiral trajectory. The through-plane (Z) data is acquired with phase-encoding. It is possible to vary the alignment of the respective lines and planes, but in order to image a tissue volume it is necessary to apply gradients and excitation and to digitize a time domain response sufficiently to encompass the three dimensional volume of tissue. This produces a three dimensional k-space matrix of data values. Processing by Fourier transforms establishes the spatial domain matrix of data that is displayed as voxel brightness or gray scale.

### SUMMARY OF THE INVENTION

In a study of a time changing image, such as breast tissue during a process comprising perfusion with a contrast agent, multiple volumetric images need to be acquired. Each of the images is like a stop frame in a moving picture. The changes from one image to the next image reveal differences tissue type and also time changes as the contrast agent is taken-up by tissue and then fades away.

It would be advantageous to provide numerous such stop frames at closely spaced points in time, so that fine distinctions can be drawn based on time. However, the time that is needed to collect and process a complete volumetric image would seem to present a minimum time limit on the time between stop frames. It is an object of the present disclosure to go below this timing limitation. A progression of images that is useful for time-changing image studies (such as contrast agent perfusion) is provided wherein a full data set is collected by fully populating the k-space data matrix initially (which can be Fourier transformed to generate a spatial image), and then producing one or more updated k-space data matrices (each of which can generate another spatial image) by populating only part of the k-space data matrix and re-using the previous values for the subset of k-space matrix values that are not updated.

It is an aspect of this technique to recognize that although the k-space matrix is a data format in the time domain and not a spatial domain, the information contained in the three dimensional k-space matrix is different for points in the matrix that are nearer to a center of the k-space matrix versus points that are spaced from the center are occupy positions near the periphery of the k-space three dimensional matrix. The k-space matrix positions near the center are related primarily to contrast. The k-space positions at the periphery are related primarily to spatial resolution. In an application such as a contrast agent perfusion study, the tissue structures do not move, but the MR response changes with changes in local concentration of the contrast agent. Advantageously therefore, the central volume of k-space is repopulated repetitively to generate a new image by Fourier transforming a version of k-space wherein the values at the central “volume” of the k-space matrix are freshly updated and the values at the peripheral volume are re-used one or more times without being updated.

In a contrast study, multiple 3D data sets are acquired to form a series of volume images at successive points in time so as to reveal differences in the local concentration of a contrast agent as the agent is taken-up by tissue and then fades away. Tissues are characterized by their dynamic (time changing) courses of contrast enhancement.

According to an aspect of the disclosure, the k-space matrix as a whole is regarded as having two parts: a central portion and a peripheral portion. The central portion is defined as k-space data positions near the origin or center point of the k-space matrix. The peripheral portion is defined as k-space data away from the origin. It is possible to set the dividing line between the central and peripheral portions more or less strictly, e.g., at a midpoint halfway between the origin and the periphery; or at a point that favors a fast frame rate, e.g., at 25% of the span between the origin and the periphery; or at a point that is somewhat slower but is less apt to be affected by motion, e.g., at 75% of the span from the origin to the periphery.

It is an aspect of MR imaging that the central portion of a three dimensional k-space data matrix when subjected to Fourier transformations contributes low spatial resolution information, particularly contrast, to the volumetric image. The peripheral portion of the k-space data matrix, when transformed, contributes the high spatial resolution information, namely spatial image detail, to the volumetric image. The division between the central portion and peripheral portion of the three dimensional k-space matrix can be made in different ways. The central portion of a cubic k-space matrix can be a smaller cube, a sphere, a polygonal solid, etc. A strict border surface can be defined, or an irregular surface.

The foregoing discussion concerns updating a central volume in k-space and retaining the peripheral k-space data to be re-used, i.e., to make a three dimensional distinction between the central portion and peripheral portion of k-space. It is also possible to employ a two dimensional planar or one-dimensional linear division. A three dimensional distinction is the general objective but two or one dimensional divisions can be employed as special cases of a 3D division. For example, a one dimensional division according to the inventive technique can be schemed to involve selecting a central zone along the slice phase encoding direction kz while having full k-space data in the kx-ky plane. Spiral imaging can also be schemed to have 2D division according to this technique: with the central portion defined as the radially inner portion of the kx-ky plane while having full k-space data coverage along the slice phase encoding direction kz.

Data is acquired as subsets of k-space data at successive time points or stop frames. Two complementary k-space data subsets for a time point can consist of a data subset for the central portion of k-space and a data subset for the peripheral portion of k-space. According to one technique, the central portion subset is populated with newly acquired k-space data to fill the defined central portion of k-space for each time point. The k-space values for the peripheral portion subset can be re-used one or more times from an earlier acquisition. For example, the peripheral portion subset can be obtained once and re-used through a full course of plural time points during which the central portion subset is updated for every time point. According to an alternative embodiment, the peripheral portion subset of k-space can be filled partially, for different matrix positions during each new time point, so as to update the whole peripheral portion over multiple time points. This can be carried on while updating the whole central portion for every time point.

Likewise according to one embodiment, the peripheral portion of the k-space matrix can be further divided into sub-divisions, for example progressively larger zones of a predetermined thickness, updated according to a schedule wherein the more central portions are updated at one frequency, preferably a higher frequency in a contrast study, and the more peripheral portions are updated at progressively lower frequencies as a function of their respective distance from the origin. The peripheral subsets acquired at different time points all are “synthesized” into at least one complete peripheral portion of k-space data, used together with the complementary central portion of k-space during Fourier transformation to generate a spatial image for each update of the central portion at the highest frequency of updating.

The digitized data collected during MRI imaging are stored in a data memory with a memory addressing scheme that organizes the digitized data as representing the k-space matrix of values. After a scan sequence, the collected data are processed through the Fourier transforms necessary to decode the magnetic moment spin density distribution in frequency and phase coordinates, producing image data representing the distribution of magnetic moment spin density. Over successive data collection cycles, the magnetic moment spin density distribution is collected and transformed to spatially distributed voxel data points at different points in time.

An important application of the disclosed technique is the diagnosis and treatment of breast cancer. By distinguishing tissue types based on their component elements or molecules, for example distinguishing concentrations of fat from concentrations of water, distinctions can be drawn to enable visualization of internal breast tissue structures, such as ducts and vasculature. Rendering fatty tissues transparent in a volume projection and enhancing water concentrations tends to highlight and impart contrast to the appearance of lesions in the images, helping a practitioner distinguish cysts from tumors, and so forth. Perfusing tissues with contrast agents improves the extent to which pertinent tissue types and tissue structures can be distinguished. Contrast agents assume different concentrations in different tissues, and may diffuse at different rates over time. A contrast agent with distinct nuclear magnetic characteristics can be injected. During and after perfusion, different concentrations of the agent in different tissue types tends to limn the contours of such tissues. By acquiring successive images over time, it is possible to compare the rates of diffusion of the contrast agent in distinct tissues.

Full MRI images typically require approximately three minutes to proceed through a full scan as needed to populate k-space fully and to generate one complete image by Fourier transform to a reasonable voxel resolution. At that rate, there may only be a few full images available in a perfusion study for meaningful comparison before the effect of the contrast agent fades away. It is an aspect of the present disclosure that associating subsets of different image acquisitions that are separated in time and/or obtained substantially from central versus peripheral zones in the k-space matrix. Re-using peripheral k-space data and/or updating the peripheral data less frequently than the complementary central k-space data, enables time changes in contrast to be monitored over incremental time samples that provide valid contrast information over a sample time that is shorter than the sample time necessary to collect full images.

It is generally necessary in MRI diffusion studies to reach a compromise between the number of images collected and the voxel resolution of the images. However the disclosed techniques provide a method for obtaining contrast information at a faster rate or in a greater number of time samples, to be used together with resolution information obtained at a slower rate, or only once during a sequence. The method exploits resolution information collected for the peripheral portion of the k-space matrix that remains valid, provided that the tissue sample remains stationary. The method enables a display of contrast and the changing concentration of a contrast agent binding preferentially to tissue structures of interest, at favorably short sampling times.

In one embodiment, a method for improving the effective time resolution of an MRI is provided. A plurality of MRI image data sets are collected over a period of time. Each of the data sets is made from plural applications of RF excitation pulses followed by sensing of responses after a period of time for populating values in k-space. The plurality of collected data sets are separated into data sub-sets, comprising earlier and later data collection sequences and comprising complementary subsets of values at central and peripheral portions of a k-space data matrix. The complementary subsets are Fourier transformed to provide volumetric image data in a spatial domain.

At least one of the data collection sequences contributes k-space data values that are spaced from the k-space origin, providing spatial resolution information. This can be a first of the sequences or repetitively according to a schedule. Preferably for each new spatial image to be generated or at least at a higher frequency schedule, complementary k-space data values are obtained during the same or additional data collection sequences. The complementary values are at and near the origin of k-space, providing contrast information. The more-frequently acquired contrast information and the less-frequently acquired (or one-time only) resolution are complementary portions that fill the k-space data matrix. Both are Fourier transformed, thereby generating images with time-spaced contrast information but at least partly sharing the spatial resolution information.

In an exemplary embodiment, a magnetic resonance imaging system includes a biasing field magnet and an array of gradient field magnets; a radio frequency pulse source that is controllable; a radio frequency receiver containing a digitizer; a control system operable to apply a magnetic field via the biasing field magnet and the gradient field magnets and to trigger application of a pulse sequence via the radio frequency pulse source. One or more processors are included, coupled to the radio frequency receiver collects digitized data values.

The processor is configured to collect a plurality of data sets corresponding to an image. A set of peripheral k-space values is collected at least once, and central k-space values are collected repetitively. The combination of central and peripheral values amounts to a full k-space data value population. By substituting new contrast information for the central part of the full k-space data set as central values are collected, and then transforming the full k-space data including the substituted contrast data, additional image-space renderings (voxel data sets) are produced compared to the number that would be possible if full k-space data sets were collected repeatedly.

### BRIEF DESCRIPTION OF THE DRAWINGS

There are shown in the drawings certain illustrative embodiments of the present subject matter; however, it should be appreciated that the invention is not limited to the embodiments disclosed as examples and is capable of variations in keeping with the scope of the subject matter defined in the appended claims. In the drawings,

FIG. 1 is a schematic view of an exemplary nuclear magnetic resonance imaging system configured for breast imaging and including a block diagram illustrating basic functional elements;

FIG. 2 is a schematic illustration equating image data in k-space and in image space, and illustrating an example of a cubic distinction between central and peripheral k-space;

FIG. 3 is a block diagram illustrating the aspect of creating image data sets by associating subsets of full image scans, in this case demonstrating the association of the later-acquired data in one sequence with earlier acquired data in a next sequence;

FIG. 4 is a block diagram corresponding to FIG. 3 but using different subsets in association; and,

FIG. 5 is a schematic illustration showing application of the subset concept of FIGS. 2 and 3 to a special case of k-space spiral trajectory MRI sequences.

### DETAILED DESCRIPTION

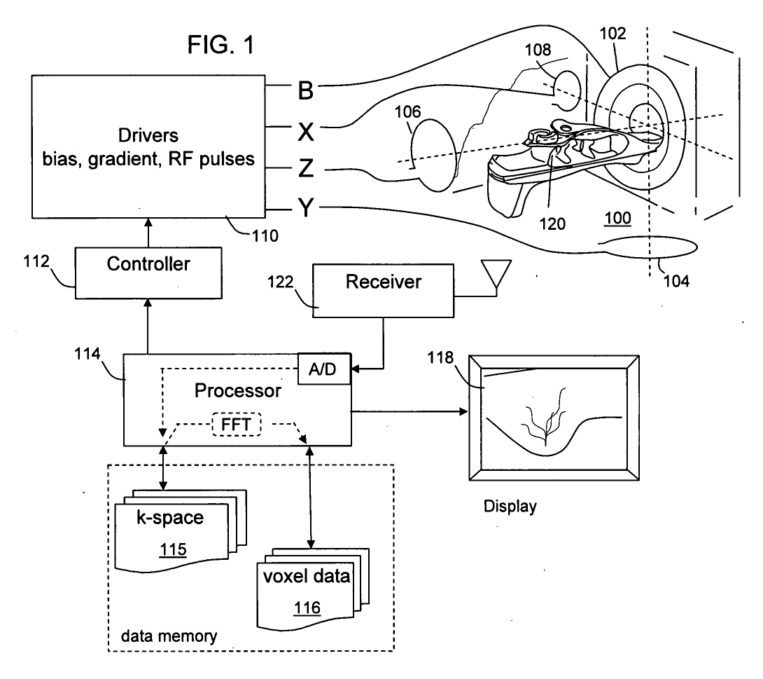

FIG. 1 shows generally the elements of a nuclear magnetic resonance (NMR or MRI) imaging system. In one embodiment, the imaging system can be a breast imaging system operated with rotating off-resonance excitation at frequencies chosen to distinguish water-based tissues while limiting the response of fat-based tissues. The system is configured to collect nuclear magnetic resonance information in a sequence of excitation and sensing operations that occurs while gradient magnetic fields are adjusted. A sequence is executed comprising excitation and phase encoding RF pulses. Each excitation is followed after a delay by a sensing interval during which the responsive signal from the imaged tissue is received, digitized and the results are stored in a data memory wherein digitized values are organized to populate a matrix conventionally known as k-space. As the sequence is executed, more and more of the image data is collected until data characterizing the response of the full tissue volume has been collected. At that point, the image data processor effects a conversion of the collected data by Fourier transforms wherein the received and digitized data is converted into a spatial map that represents the localized amplitude of magnetic resonance response versus position in the tissue volume. The amplitude values are projected as voxel values in a volume or pixel values projected onto a display.

The data for one or more excitation sequences is collected over a period of time during which RF excitation pulses followed by sense/digitize operations are repeated. It is an aspect of the disclosure that data is collected over a period of time to yield a series of stop frame images at discrete points in time. It is also an aspect, however, that each stop frame does not require a complete image collection sequence. Later collected data, specifically representing contrast information, is processed together with a complementary set of data that represents resolution but is at least partly re-used from excitation and sense/digitize sequences at different times. The later collected data and the re-used data are complementary portions of the digitized data in k-space.

It is an attribute of k-space data that values nearer to a k-space origin are relatively more associated with contrast. Values radially spaced from the k-space origin are relatively more associated with resolution. An aspect of the present technique is that while collecting data over a period of time, portions of k-space data sets associated with contrast are collected repeatedly and are associated with those portions of at least one k-space data set that are associated with resolution. The invention permits contrast information from a later time to be substituted for the contrast information of from a previous time, while re-using the resolution information.

According to an alternative embodiment, the central portion of k-space is repetitively updated and overwritten with new information. After every such update of the central portion of k-space, the full content of all of k-space is Fourier transformed to produce a voxel data set. At least once, and preferably at a repetitive rate or schedule of partial updating that is less frequent than the updating of the central portion of k-space, the peripheral portion of k-space is written. The central and peripheral portions of k-space are complementary with one another, and produce voxel data sets repetitively for every update of the central portion of k-space, even though the complementary portions of k-space were updated at different times.

This technique provides updated contrast information in the voxel data image and requires less time that a full image collection process. Provided that the tissue is stationary, the composite images derived from Fourier transformation of two (or more) complementary portions of k-space retain resolution and detail, and the contrast information is updated according to an advantageously short cycle time. The disclosed technique is apt for diagnostic procedures that have a time-changing contrast aspect but wherein the tissues themselves are stationary, such as a procedure wherein tissues are perfused with a contrast agent and then repeatedly imaged to assess the changing luminance of the contrast agent as the agent diffuses at different rates in different tissues. Aspects of the invention are also applicable to other applications, other tissue types and other pulse sequences.

The imaging system as shown in FIG. 1 comprises a set of electromagnets including a biasing coil 102, establishing a static magnetic biasing field, B₀, in a longitudinal direction with respect to a patient (not shown). For breast imaging, the patient lies prone on a supporting table 120 with breasts depending and optionally held in one or more positioning fixtures (not shown). Table 120 can be translated in an axial direction relative to biasing coil 102 to move the patient into and through the lumen of coil 102 for imaging the breasts and anterior torso. In one application, the imaging can be conducted according to a spiral RODEO sequence in an X-Y plane and by phase encoding on the Z-axis (wherein the Z-axis corresponds to the head-to-to direction of the patient), as provided by Aurora Imaging Technology, Inc., North Andover, Mass.

The static magnetic field of biasing coil 102 as shown is aligned in the longitudinal head-to-toe direction relative to the patient, which can be regarded as the Z-direction. Additional magnetic coils 108, 104 are positioned to apply variable magnetic field gradients in the orthogonal X- and Y-directions, respectively. Also, a phase-encoding coil 106 is provided with an orientation parallel to that of the biasing coil 102 for applying an excitation pulse. A read antenna is coupled to a receiver 122 for sensing the signal, which is amplified, time sampled and digitized, and stored. The sample data is arranged in a memory that can be organized according to a k-space coordinate system. When sampling and digitizing is complete, Fourier transforms convert the time domain sample data to spatial domain voxel data.

The biasing coil field causes atoms in the patient's tissues to be aligned to a reference spin orientation. The fields produced by the gradient coils are varied so as to select a local area to be imaged in the patient tissue. The gradient fields preferably are varied in a periodic manner so as to encode and select, one after another, successive lines or planes in the tissue. The spin axes of the atoms that are addressed are displaced from the reference orientation determined by the biasing field. RF excitation pulses are applied. Excitation at a predetermined Larmor frequency that is resonant for a given element encodes a phase coherent magnetic spin in atoms of that element. The atoms precess in phase for a time, eventually become phase incoherent and eventually return to the reference orientation determined by the biasing field. Time domain sample data is collected, digitized and stored in the k-space memory. After proceeding through one or more sequences that progress through the volume of tissue, the processor 114 Fourier transforms the contents of k-space memory 115, thereby producing voxel data values that are stored in memory locations of a voxel memory 116, from which slice or projection displays can be generated and presented on display device 118.

After the RF excitation pulse, the magnetic spins of atoms in the local area being imaged are in phase until they become phase incoherent over a “t2” relaxation time. The spins fade away over a “t1” relaxation time as the precessing atoms return to the reference spin orientation determined by the biasing field. The t1 and t2 times are specific to the element excited at the frequency of the RF excitation and the relaxation times can provide a way to distinguish among different concentrations of elements.

In order to accomplish excitation and sensing of an area or volume of tissue using a succession of excitation pulses and echo sensing and encoding steps, it is necessary to set a predetermined gradient field strength, apply an RF excitation pulse, and encode the resulting signals in a coordinated manner, moving from point to point. A controller 112 is coupled to be driven by signals from a computer processor 114 and in turn triggers operation of the gradient and excitation drive apparatus 110. This drive apparatus 110 can also be the source of a steady stage current to drive the bias coil 102.

In this embodiment, bias and gradient drive apparatus 110 applies a timed sequence of pulses in coordination with varying the current in the X-Y-Z gradient coils 108, 104, 106. In time with the application of the excitation and gradient pulses, the computer processor 114 obtains the echo response of the patient tissues via receiver 122.

After a sequence of pulses has been executed sufficient to obtain a full dataset, the processor computes from the k-space time domain data array a corresponding spatial domain image data array. The results are stored as digital amplitude values according to physical position coordinates in real space, i.e., to an array of X, Y and Z points in the space occupied by the imaged tissue.

This image data can be stored in a voxel data memory 116. Using volumetric image data processing, it is possible to select an arbitrary slice through the imaged volume for display. The data can be processed to obtain a two dimensional projection of the three dimensional volume, for example including rendering some of the detected tissue types as transparent so as to reveal other tissue types. This projection can be rotated, zoomed, etc. For breast imaging, tissues with fat concentration can be rendered as transparent to better visualize ducts and potential lesions with water concentration.

The data can be processed and enhanced using image processing software, for example to adjust contrast. The image data can be combined by addition or subtraction or Boolean function with other images of the same volume, subjected to threshold detection, etc. The resulting images preferably are displayable on a display apparatus 118, enabling a physician or clinician to visualize internal tissues.

It is an aspect of the invention that instead of collecting complete k-space data sets one after another, the contrast portions of a data set are collected one after another but the resolution data in one or more data sets is re-used by updating the corresponding portions of k-space memory according to different schedules. In one embodiment, after collecting a complete data set in k-space and generating an image by Fourier transform, an updated image is generated using one or more further image collection steps that update only a part of the data set in k-space, namely the contrast information corresponding to a zone or volume adjacent to a k-space origin, i.e., the central portion of the three dimensional matrix of k-space values. The contrast information at least is collected more frequently than the resolution information. According to one embodiment, the resolution information (peripheral portions of k-space) is collected once. Contrast information (central portions of k-space) is collected over several repeated sequences. A Fourier transform and new voxel data image can be generated for every update of the contrast information, using the complementary new contrast information and re-used resolution information stored in k-space memory.

Rather than being re-used indefinitely, the resolution information at the peripheral portions of k-space can be updated at a less frequent schedule than the contrast information at the central portion. For example, over a given number of sequences during which contrast information is collected, for example ten sequences, only a corresponding proportion of the resolution values (i.e., one tenth in this example) might be updated. Each of the more-frequently collected inner portions of k-space showing contrast can be used to compose an individual image for display, wherein the synthesized composite of partially updated parts of the less-frequently collected outer portions of k-space are used to complement the inner portions. Together, the portions provide a fully populated k-space matrix, although portions of the matrix were collected at different times. After Fourier transform, the full resolution of the image data set (i.e., the full complement of pixels at the finest resolution) is obtained but the contrast data has a sample time resolution that is shorter than would be possible if it was necessary to collect complete datasets anew, for Fourier transformation and display one after another.

Processor 114 can apply various image processing steps to the voxel data stored in voxel data memory 116. Without limitation, such steps can include enhancement of contrast by edge detection, threshold level discrimination, application of pattern enhancement masks, image analysis transforms, and the like. Processor 114 is configured to collect plural images of the same volume before and after one or more processing steps. These images are applied to one another such that voxels in registry are added or subtracted or subjected to thresholds and Boolean operations, in each case to provide different techniques for producing contrast.

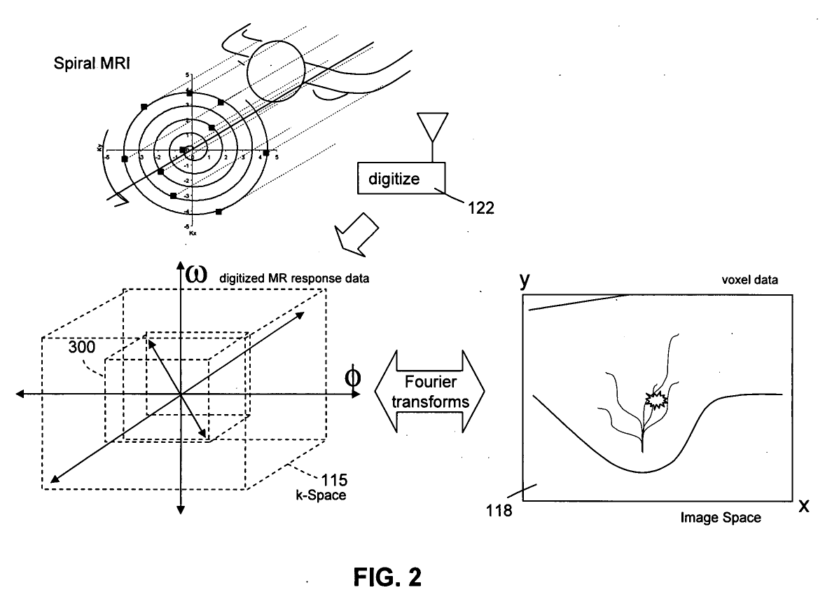

The NMR imaging arrangement illustrated in FIGS. 1 and 2 may be configured to employ a spiral “RODEO”. The acronym “RODEO” denotes “rotating delivery of excitation off resonance.” In a spiral RODEO three dimensional imaging process, gradient field modulation is arranged for the acquisition of k-space time domain data while proceeding along a spiral in the kx-ky plane and phase encoding along the kz axis. The preferred RF pulse can be arranged to excite water protons to produce fat-suppressed images. The particular pulse sequence produces fast T1-weighted images that proceed in a spiral while collecting data for k-space. It is an advantage that good biasing-field (B₀) homogeneity is maintained across the imaging field-of-view during spiral scanning. Tight specifications are preferably applied on shimming and eddy current compensation performance.

In FIG. 2, a volumetric image of the patient is to be rendered in image space. The MRI controller sequences through gradient, excitation and phase encoding steps, delay, sensing, digitization and storage of responsive time sampled values in k-space. Referring also to FIG. 1, the response is sampled in time by receiver 122 and digitized, the results being stored in a k-space memory 115, of which a subset 300 represents contrast information. The contents of the memory 115 are Fourier transformed to render an image for display 118. In FIG. 2, the central subset 300 of k-space 115 represents contrast information and the complementary remainder of k-space 115 is the peripheral portion containing resolution information. As discussed, the division could be according to a proportion other than half the dimension on a side (one eighth the volume of k-space) as in the example shown, and could be according to a geometry that is other than cubic, e.g., spherical of according to another shape. Likewise, the surface distinguishing the central and peripheral portions can be irregular and it is possible to provide more than two zones that are respectfully relatively more central or peripheral and are updated on different schedules.

At least the most central portion (300 in the example) is overwritten with new data from a subsequent sequence or from every successive sequence. The peripheral remainder of the contents of memory 115 in k-space, complementary to the central portion, is reused, being obtained only once or updated less frequently than the central portion. A new image is obtained using the new contrast data in subset 300 and re-using the resolution data from the peripheral portion.

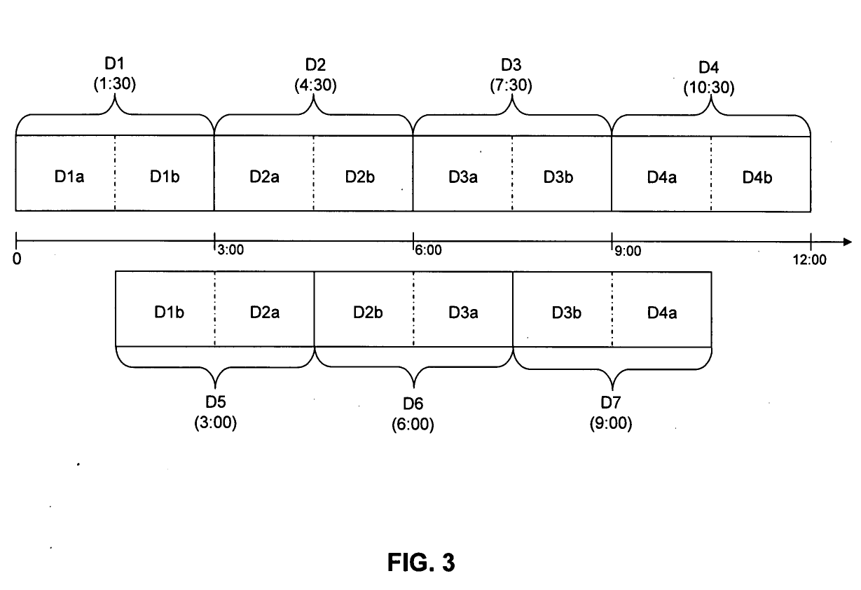

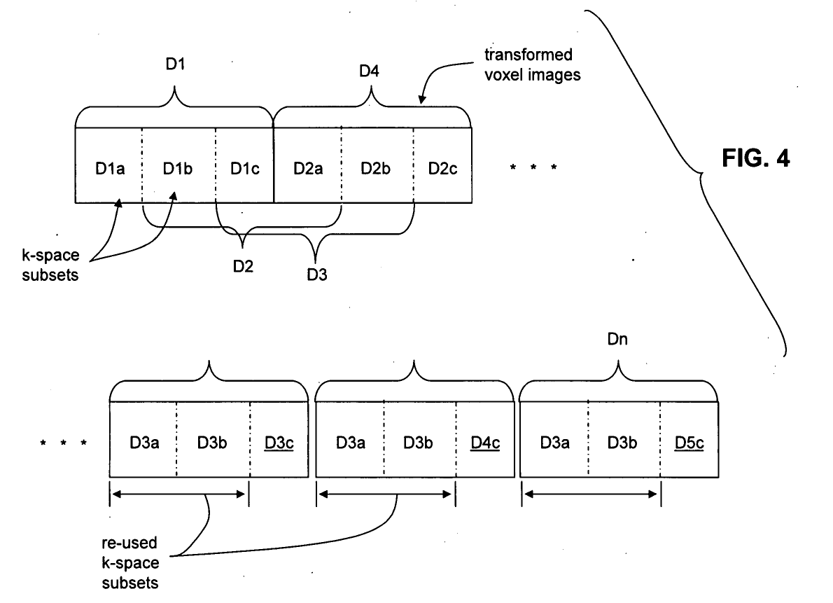

FIGS. 3 and 4 demonstrate some exemplary alternatives for combining subsets of k-space data, preferably collected sequentially, to produce a number of images from combining central and peripheral portions of k-space data that are collected at different times. This is accomplished by Fourier transformation of a k-space dataset after substituting the values of the most recently collected subset for the previous data values in k-space. It is also possible in this way to provide more images than updates, by producing a Fourier transformation and a distinct image from different sets of associated subsets.

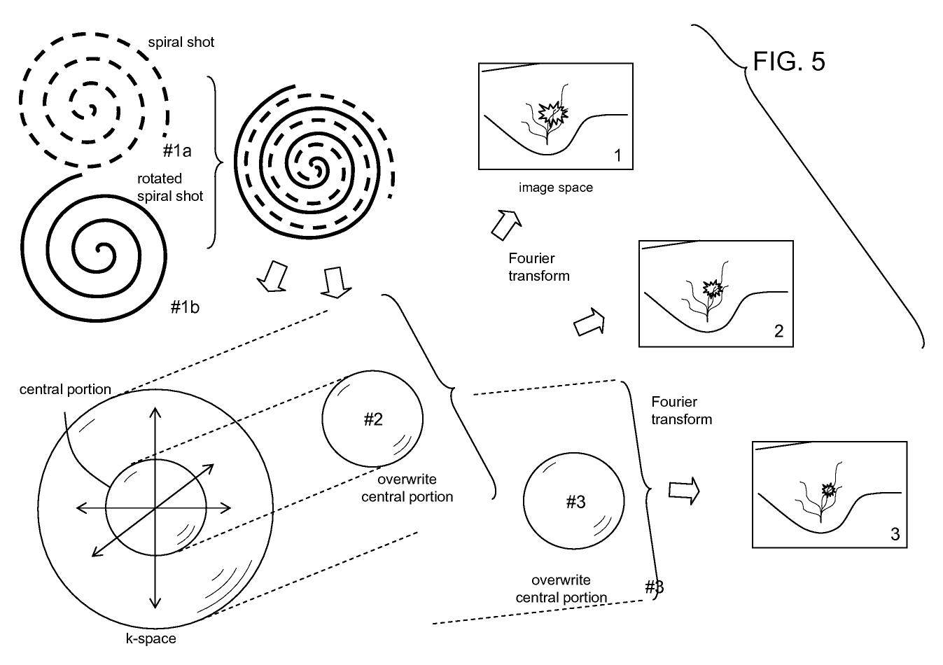

In one embodiment generally shown in FIGS. 2 and 5, the pulse sequence design that is executed (i.e., the planned timing and sequence of excitation and gradient pulses), comprises a slew rate-limited spiral trajectory gradient waveform, applied repetitively to collect k-space values for each point in a trajectory or spiral “shot”. From one spiral trajectory to the next, the pitch or rotational origin or centering of the spiral pattern is varied so that successive spirals progressively fill in the points in the imaged volume. An example is shown in FIG. 5, wherein spiral shots 1a and 1b are relatively rotated such that the positions of points in the X-Y plane do not overlap as phase-encoded tissue segments parallel to the Z axis are excited and their MR response received and digitized. The data collected during the successive spiral shots populates k-space.

The multi-shot interleaved trajectory can be implemented by rotating a matrix multiplier applied to the gradient in the pulse sequence programming. Multi-shot spiral imaging requires plural scans and may require a longer total scan time than a single-shot spiral if used to collect a full image data set. However one or more spiral shots can be collected to update an image in time.

According to one arrangement, the pulse sequence comprises a RODEO RF pulse (described further below), followed by off-centering gradients to displace the present sensing position along the kx and ky axes, and a phase-encoding gradient to progress along the Z-axis. At the end of a spiral, readout-rewinding gradient pulses are applied to all three axes to reset the nuclear spins. A spoiler-gradient pulse can be applied along the Z-axis. The spoiler-gradient pulse seeks to desynchronize and randomize any residual nuclear spin.

According to one embodiment, the sequence uses a RODEO RF pulse that comprises two back-to-back cosine shaped pulses at a frequency centered on the resonance frequency of atoms concentrated in fat tissue. The first cosine shaped pulse, extending from 0 to 2π radians, centers on the fat resonance frequency. This RF pulse is immediately followed by a similar cosine shaped pulse having the same period, amplitude, and frequency as the first pulse, but with a 180° phase shift. The combination of the two cosine-shaped phase-reversed pulses results in cancellation of on-resonance spins and thereby suppresses the fat response signal in the collected data images. At the same time, those two pulses are additive for off-resonance spins. Water is off-resonance where the cosine-shaped pulses were made resonant to the fat signal that is suppressed. As a result, the RODEO pulse sequence suppresses the image of fat-resonant portions of the collected data image, and improves the contrasting image of the non-fat resonant portions, including concentrations of water and tissues with relatively low fat concentration such as ducts.

The image reconstruction from the spiral k-data is implemented using an algorithm of non-uniform Fast Fourier Transformations (FFT). This method generates the 2D gridding kernel matrix for a given spiral trajectory using a least square approach. Specifically, the reconstruction process consists of the following steps:

apply 1D FFT along z axis on acquired data;

generate the kernel matrices corresponding to the spiral trajectory;

grid k-data by convolving spiral k-data with the kernel matrices;

perform filtering and 2D FFT on gridded k-data; and

rescale and format the images.

A one dimension FFT is applied in the slice direction for each of the two dimensional k-space data points. This process allows zero-fill upon reconstruction parameter request.

The spiral RODEO imaging technique described above is apt for diagnostic diffusion studies. The object of a diffusion study is to provide a time plot in which the onset and fading away of contrast due to the perfusion and diffusion of a contrast agent are noted over time, and differences in the fading of the contrast agent tend to distinguish lesions or tumors from other tissues such as cysts. In such procedures, a preliminary base image is collected prior to the application of a contrast agent to the patient. Once the base image is collected, the patient is given a predetermined amount of a contrast agent. Gadolinium-based contrast agents are typically used as they are paramagnetic compositions that tend to concentrate in lesions and enhance the contrast of the lesion in the collected image.

Subsequent to the patient receiving the contrast agent, one or more full resolution images are collected to fully populate k-space with image data. Subsequent imaging passes are then made, for updating and overwriting the central portion of the k-space dataset as shown in FIG. 5. These subsequent passes are distributed over the time it takes for the contrast agent to diffuse in the patient's body, and can be made one after another. Each time that a pass is completed to update the central portion of k-space, the complete k-space dataset including the updated central portion and the pre-existing data for the peripheral portion, is available to be Fourier transformed to produce a dataset in voxel image space. Insofar as the central part of k-space can be overwritten in less time than it would take to populate all of k-space with new data, the technique provides images with changing contrast information more frequently and in a larger number than would otherwise be possible.

A plurality of MRI image data sets are collected over a period of time. The collected data sets are separated into data sub-sets, comprising earlier and later data collection sequences and comprising complementary subsets of values at central and peripheral portions of the k-space data matrix. The complementary subsets are Fourier transformed to provide volumetric image data in a spatial domain.

It is possible to employ a schedule wherein the central and peripheral parts of k-space are both overwritten, but at a different frequency. Preferably, the contrast information from the central part of k-space is updated most frequently. One or more relatively more peripheral parts of k-space are updated less frequently, or not at all.

At least one of the data collection sequences contributes k-space data values that are spaced from the k-space origin, providing spatial resolution information. This can be a first of the sequences or part of a repetitive process according to a schedule. If the Fourier transforms are accomplished after the sequences are all completed, it is also possible to use resolution information in peripheral k-space from a later sequence, instead of an earlier sequence, together with the central k-space subsets from earlier sequences, i.e., to collect the peripheral k-space subset at any time in the succession of imaging shots. The processor is configured to collect a plurality of data sets corresponding to an image, at least one of which contains at least one set of peripheral k-space values, and wherein a complementary set of central k-space values are collected repetitively. The voxel images are obtained by Fourier transforming combinations of these complementary sets.

As a simplified example, if a contrast agent fully diffuses in a patient's body in ten minutes, and a full multi-shot spiral RODEO imaging pass takes five minutes to achieve the desired resolution, a maximum of two full imaging passes might be made within the allotted time, resulting in two images or two values for any particular spatial voxel position. However according to the invention, after collecting a full image data set, it is possible to update the data set repetitively with a subset of central k-space values. For each update a new image and new value is possible. If, for example, a limited central k-space subset can be collected in one minute, then five updated images can be generated by successively updating only the central k-space data subset and Fourier transforming the full k-space dataset to produce a new image.

The inventive technique comprises over-writing, substituting or similarly associated in a k-space memory, the collected values for a subset of collected data values that make up a pre-transform image in k-space, one or more other subsets that are complementary and together with the while not over-writing a different subset of already-stored values existing in the k-space memory. Then, Fourier transformation converts all the stored k-space values from MR response into voxel values (e.g., luminance) as a function of spatial position. This provides a combined or hybrid image made up in part by the values for the subset of points, and in part by the values already stored in k-space that were not overwritten.

Referring to FIG. 3, in one embodiment, the values used to over-write the existing values are values that are collected later in time than the existing values, and thus the Fourier transformation produces a new and partly updated complete image. In the example of FIG. 3, each image is subdivided by time of collection, in this example by half. Thus, each image Dn contains two subsets Dna and Dnb that respectively fill half of the MR value coordinate positions in k-space memory. Four images D1 through D4 are produced by associating each pair of image subsets together. However, it is also possible to substitute the subset of different images to provide additional full images. If the image subsets are consecutive in time as shown in FIG. 3, then in addition to images D1 through D4, three additional images D5 through D7 are possible by associating the later parts of the earlier data collection sequences (Dn_{b}) with the earlier parts of a next later sequence (Dn+1ₐ). Each associated collection of two subsets in this example contains a full set of values for all of k-space, and thus can produce a voxel image by Fourier transformation. This technique can be used whether or not there is also a division between the central k-space subset containing contrast information and the peripheral k-space subset containing resolution information.

The technique of associating earlier-collected and later-collected complementary subsets of k-space values is useful in diffusion studies wherein the contrast produced by a perfused contrast agent dissipates over a time as the contrast agent diffuses in stationary tissues. In FIG. 3, where there are two subsets used, it is advantageous to collect a full image D1 containing subsets D1a and D1b, of which one D1a is the peripheral part of k-space (resolution information) and the other D1b is the central inner part 300 of k-space (contrast information) as shown in FIGS. 2 and 5. Then, subsequent images Dn are generated by repeated substituting only the central part of k-space, Dnb, and providing new Fourier transforms for each substitution.

The technique is applicable to other subsets, such as providing repetitive incrementally rotated spiral shots in k-space wherein a predetermined number ‘m’ will fill the k-space memory, and repetitively generating Fourier transformations to produce images from a collected subset of the shots numbering between one and ‘m’. FIG. 4 shows an embodiment wherein three shots produce an image. After collecting a full image D1 from shots producing subsets D1a, D1b, D1c, a moving substitution can produce successive images that are updated as to one, two and then three (all) subsets in k-space, providing images D1 through D4. As another alternative, one or more subsets D3a, D3b can be re-used by overwriting subset D3c, with subsets D4c, D5c, etc. Although in these examples, the new subsets are collected sequentially, it is possible to combine subsets in different orders as well.

FIG. 5 illustrates certain embodiments wherein k-space data is populated using spiral scan shots in k-space, each spiral being rotated relative to a previous one so as to fill the area between the spiral arcs with the arcs of the next spiral. Assuming that all the necessary data points in k-space are filled using some number of scan shots in one or more sequences (#1a and #1b, for example), the data when sampled and digitized fills a k-space memory in three dimensions. A Fourier transform of k-space generates image 1.

In a next sequence, only a central part of k-space is digitized and stored. However that central part together with the complementary peripheral part of k-space contains a full k-space data set. This full k-space data set is Fourier transformed to produce image 2. Then a new central part of k-space is provided to substitute for the existing data, Fourier transformed and the process is repeated.

The over-writing of subsets of data values occurs in k-space. As a result, the effect of over-writing and generating a new transform is to update aspects across the whole image as opposed to updating particular voxel data positions in an image memory (such as might characterize the interleaved scanning of a video raster).

The invention is not limited to use in the display of images updated by subsets in k-space. Various image processing and image comparison steps can be taken as well, either separately or in conjunction with generating new images, and involving one or more of the previous images or subsets in k-space or in voxel space. In a diffusion study, for example, a desired number of post-contrast imaging passes can be made and their subsets stored before generating transforms from the combination of their subset data with remaining image data collected either previously or subsequently. A pre-contrast image in voxel space can be subtracted from the post-contrast image to enhance changes in contrast, thereby darkening fluid and edema images in the tissue enhancing the high-contrast lesions in the displayed image. A practitioner may study the diffusion of the fluid in the patient's body over time as each of the enhanced images or subsets represents a different point in time. Although it might be possible during the time of diffusion to collect only a few full images, the practitioner can exploit the available time to provide contrast updated images to focus on how the contrast agent diffuses in the body of the patient.

In accordance with an exemplary embodiment, the present technique provides a method to enhance the information made available in a magnetic resonance imaging procedure by substituting subsets of data in k-space, and Fourier transforming the data to produce images that use a full population of k-space values (once a full k-space image has been stored) but only some of the values are from newly substituted subsets of the population. The images are generated by transforming combined subsets, each of which may be distinct in one or both of time and area of k-space populated by the subset.

The invention can be provided to generate image data sets one after another by overwriting a subset of values in k-space memory. Preferably, however, the imaging data from a base reference image is stored and then one or more subsets of data values are stored separately such that it is a matter of programming to associate different k-space subsets to generate hybrid images. Advantageously, the process can proceed as a sequence selected by the operator, for example comprising a schedule for repetitive collection of a base or reference image followed by collection of one or more subsets in k-space and Fourier transformation to produce one or more images built from the k-space data of the reference and the subset(s).

It is not necessary to collect a full dataset populating all of k-space before transforming the data to produce a next distinct image. If full k-space data has been filled at least once during the process of repetitively collecting k-space data subsets, the information from new data can be associated with resolution information collected during the image acquisition and upon Fourier transformation produces a useful image.

In the examples to this point, and as shown in FIG. 2, two distinct zones of k-space are distinguished (inner contrast versus outer resolution). It is also possible to provide for a different number of zones, such as three zones, suggested by the example at the bottom of FIG. 4. The controller 112 can be programmed to offer selections to the operator for alternative division of k-space into subsets, alternative schedules for which subsets are collected at which points in the sequence of imaging, and whether there shall be a new Fourier transform and voxel image generated after each subset is over-written on the corresponding information in k-space.

Although the invention has been described in terms of exemplary embodiments, it is not limited thereto. Rather, the appended claims should be construed broadly, to include other variants and embodiments of the invention, which may be made by those skilled in the art without departing from the scope and range of equivalents of the invention.

What is claimed is:

1. A method for magnetic resonance imaging, including the steps of:
performing a plurality of magnetic resonance imaging passes by applying resonant excitation to a subject placed in a magnetic field and sensing and storing in a k-space memory data representing a level of magnetic resonance response;
wherein the data stored in the k-space memory comprises at least two subsets corresponding to distinct coordinate zones in k-space, and wherein the at least two subsets in combination represent a full dataset representing a magnetic resonance image;
performing at least one further magnetic resonance imaging pass and storing in k-space memory at least one additional version of at least one of the two subsets;
performing Fourier transformation to provide from the k-space memory at least two voxel images that respectively represent different combinations of the at least two subsets with the at least one additional version; and,
at least one of storing, displaying and transmitting the voxel images.

2. The method of claim 1, comprising repetitively performing said at least one further magnetic resonance imaging pass and repetitively obtaining an updated said additional version of the at least one of the subsets.

3. The method of claim 1, wherein the subsets are distinct zones in k-space comprising a central volume including a k-space origin and at least one peripheral volume disposed between the central volume and a periphery of said k-space.

4. The method of claim 1, wherein the subsets are distinct zones in k-space that are respectively nearer to a k-space origin and relatively spaced from the k-space origin.

5. The method of claim 1, comprising repetitively collecting as the at least one additional version magnetic response values for a zone of k-space that is centered on a k-space origin, and further comprising overwriting values for a full range of said k-space with corresponding data from said zone of k-space so as to substitute the additional version for a previous version of data for said zone, and generating by said Fourier transformation a voxel image of amplitude versus spatial position for a full range of said k-space.

6. The method of claim 2, further comprising applying a contrast agent to the subject and holding the subject substantially stationary during imaging, and wherein the additional versions repetitively collected as the at least one additional version magnetic response are values for a zone of k-space that is centered on a k-space origin, whereby the additional versions provide time spaced updated information respecting contrast caused by the contrast agent.

7. The method of claim 6, further combining and updating the subsets according to a sequence provided by a controller.

8. A magnetic resonance imaging system, comprising:
a biasing field magnet and an array of gradient coils,
a radio frequency pulse source;
a radio frequency receiver;
a control system operable to apply a magnetic field via the gradient coils and to trigger application of a pulse sequence via the radio frequency pulse source;
a processor coupled to the control system and to the radio frequency receiver, wherein the processor is configured to execute excitation and to collect magnetic resonance response values for populating a k-space array in data memory;
wherein the processor is configured to associate together different portions of the k-space array as subsets, whereby said subsets can be combined by the processor to fill the k-space array by occupying different said coordinates, and the processor is programmably operable to effect Fourier transformation of the k-space array;
wherein the processor is programmed to collect and store successive versions of at least one of the subsets to provide at least one additional image data set wherein values for one of the subsets has been changed compared to a previous version.

9. The magnetic resonance imaging system of claim 8, wherein the magnetic resonance imaging system comprising a spiral imaging system configured to accumulate the collected image data set using plural spiral scans.

10. A computer readable medium encoded with program code, wherein when the program code is executed by a processor for performing a method comprising the steps of:
managing data derived from a MRI imaging, wherein the data for each MRI image comprises a plurality of data subsets in k-space and contribute to distinct portions of k-space prior art Fourier transformation;
collecting and organizing the data subsets such that at least one of the subsets in k-space is substituted for corresponding previously acquired subset of k-space and said at least one subset and said previously acquired subset define a full image data set in k-space; and,
at least one of communicating, storing, Fourier transforming, and displaying the additional MRI image.

11. The computer readable medium of claim 10, wherein the method further comprises controlling a coordinated application of magnetic gradients, excitation pulses and sensing and digitizing operations.

12. A method for interpolating data points in an MRI contrast agent diffusion study comprising the steps of:
applying the contrast agent to patient tissue and fixing the patient in a substantially stationary position;
performing an MRI imaging process to provide at least two MRI images of the patient tissue, wherein each of the MRI images comprises plural data subsets in k-space that together provide said MRI images with a predetermined image resolution;
organizing the data subsets in k-space so as to combine different versions of at least one of the subsets with a same version of at least on other one of the subsets, thereby producing an image combining results of MRI scanning operations that are distinct in one of time, sequence and corresponding zone of k-space.

13. The method of claim 12, wherein the different parts of the succession that are associated extend at least one data subset of a complete reference MRI image with a radially central zone of k-space from a second data subset that is not a part of the reference MRI image.
